(19) United States
(12) Patent Application Publication
(10) Pub. No.: US 2025/0349388 A1
(43) Pub. Date: Nov. 13, 2025

(54) METHOD, APPARATUS, AND ELECTRONIC DEVICE FOR CORRECTING BASE QUALITY SCORES ACCORDING TO SEQUENCING PLATFORM CHARACTERISTICS

(71) Applicant: GENECAST PRECISION MEDICAL DIAGNOSTIC LABORATORY WUXI CO., LTD., Jiangsu (CN)

(72) Inventors: Dan LIU, Jiangsu (CN); Rui FAN, Jiangsu (CN); Yaxi ZHANG, Jiangsu (CN); Yu HUANG, Jiangsu (CN); Weizhi CHEN, Jiangsu (CN); Bo DU, Jiangsu (CN)

(73) Assignee: GENECAST PRECISION MEDICAL DIAGNOSTIC LABORATORY WUXI CO., LTD., Jiangsu (CN)

(21) Appl. No.: 19/226,075

(22) Filed: Jun. 2, 2025

Related U.S. Application Data
(63) Continuation of application No. PCT/CN2023/088611, filed on Apr. 17, 2023.

(30) Foreign Application Priority Data
Dec. 20, 2022 (CN) 202211638241.X

(51) Int. Cl. G16B 30/10; G06F 17/18; G16B 20/50

(52) U.S. Cl. CPC G16B 30/10; G06F 17/18; G16B 20/50

(57) ABSTRACT

The current application discloses a method, apparatus, and electronic device for correcting base quality scores according to sequencing platform characteristics. This method extracts the overlapping bases, distinguishes sequencing errors from non-sequencing errors, and divides the extracted overlapping bases into different bins according to the read direction where the sequencing error base is located, the number of sequencing cycles, the dinucleotide in the sequencing direction, and the base quality score given by the sequencer. The sequencing error bases in each feature bin are counted, and the empirical quality value is calculated. A lowess model is used to perform polynomial fitting modeling on the RQS and EQS in the feature bins and to correct the original base quality score. The current application can distinguish between sequencing errors and non-sequencing errors, can more accurately reflect the sequencer preference, and can conduct modeling corrections, thereby comprehensively improving the credibility of base quality scores.

### CROSS-REFERENCE TO RELATED APPLICATION

This application is a continuation of international application of PCT application serial no. PCT/CN2023/088611 filed on Apr. 17, 2023, which claims the priority benefit of China application no. 202211638241.X, filed on Dec. 20, 2022. The entirety of each of the above-mentioned patent applications is hereby incorporated by reference herein and made a part of this specification.

### TECHNICAL FIELD

The current application pertains to the domain of gene sequencing technology and specifically relates to a method, apparatus, and electronic device for correcting base quality scores according to sequencing platform characteristics.

### BACKGROUND OF RELATED ART

With the widespread application of high-throughput sequencing technology (NGS) in the field of companion diagnostics of tumors, early screening and minimal residual disease (MRD) detection based on liquid biopsy (cfDNA) have also received increasing attention and recognition. Although the continuous development of NGS sequencing platforms has provided higher throughput and sequencing accuracy, the quality of sequencing data is still a key technical bottleneck for identifying DNA (ctDNA) from tumor tissue. For example, identifying somatic mutations in cfDNA samples often requires reaching a level below 0.1%, a detection requirement that is lower than the common error rate of current sequencing platforms. How to effectively minimize sequencing errors and pinpoint genuine low-frequency variants is crucial for boosting the efficacy of detection tools.

The common format of sequencing reads produced by NGS sequencers is fastq, where each read consists of four lines. The length of the reads is determined by the sequencing mode, and each position contains base sequence (ATCGN) information and its corresponding base quality score (Q-score) information. The base quality score is an integer mapping of the probability of base recognition error, Q=−10×1g (P), where P is the probability of base recognition error. The base quality score represents the sequencer's assessment of the credibility of the base sequence signal and is a direct indicator for evaluating sequencing errors. For example, A base quality score of Q20 signifies that the confidence level in the base being a correct sequencing result is 99%. However, the base quality scores generated by the sequencer are affected by various factors, among which systematic preferences often lead to overestimation or underestimation of the base quality scores in the data, resulting in batch effects. Therefore, in order to eliminate the sequencing error preferences of different platforms and batches and to improve the applicability of detection technologies and methods, it is particularly important to correct the base quality scores of sequencing data.

At present, the most commonly used method for correcting base quality scores is GATK-BQSR. Its principle is to treat all bases that are mismatched with the reference genome outside the species mutation database as sequencing errors. For species that do not yet have a complete population mutation database, this method cannot correct quality values. In addition, the sequencing errors of GATK-BQSR include mismatches introduced before sequencing, such as sampling and library construction, biological somatic mutations, and true sequencing errors. For the cfDNA data used in liquid biopsies for disease diagnosis that currently pursues high sensitivity and accuracy, sample-specific somatic variations and mutations produced by clonal hematopoiesis cannot be ignored. The GATK-BQSR quality value correction method cannot distinguish between these sample-specific biological variations and true sequencing errors (i.e., base recognition errors), which will affect the correction results. In particular, it may cause very serious deviations for tumor samples with high TMB, thereby having a significant impact on the sensitivity and accuracy of data detection. GATK-BQSR's modeling and correction at the sample level require more time and computing resources for high-depth and large-volume data, which will extend the product data analysis cycle.

### SUMMARY OF THE INVENTION

### 1. Problem to be Solved

The current application addresses one of the issues with base quality score correction using GATK-BQSR, as previously mentioned. It provides a method, apparatus, and electronic device tailored for correcting the base quality scores according to sequencing platform characteristics. The method utilizes the original double-end sequencing data, extracting the overlapping bases from read1 and read2. It employs these overlapping bases to differentiate between sequencing error bases and non-sequencing error bases. The bases within the extracted overlapping region are categorized into various bins based on the read direction (read1 or read2) of the sequencing error base, the number of sequencing cycles (cycle), the dinucleotide in the sequencing direction, and the report quality score (RQS) provided by the sequencer. The method counts the erroneously sequenced bases within each feature bin and calculates the empirical quality score (EQS). It then applies a locally weighted regression model (Lowess) to perform polynomial fitting and modeling on the RQS and EQS within the feature bins. Finally, it uses the resulting model to correct the original base quality scores. The current application can differentiate between bases that are sourced from sequencing errors and those that are not, more accurately reflecting the preferences of the sequencer. Based on this, the accuracy of the base quality scores provided by the sequencer is evaluated, modeled, and corrected, which can comprehensively enhance the reliability of the base quality scores.

### 2. Technical Solutions

To address the aforementioned issues, the technical solutions implemented in this application are as follows:

As the first aspect of the current application, it provides a method for correcting base quality scores for sequencing platform characteristics, the method comprising the following steps:
S1: The raw double-end sequencing data is preprocessed and aligned to the reference genome to generate a Bam file. The preprocessing includes removing the adapter sequence and low-quality sequences.
S2: Using the Bam file generated in S1 as the input file, a consensus sequence of the reference genome is constructed and reads-level filtering is performed, but the bases in the overlapping part of read1 and read2 are not filtered according to the base quality score, that is, the bases in the overlapping part of read1 and read2 are retained from the filtering process;
S3: Extract all overlapping bases of reads pairs (i.e., read1 and read2) from the filtered Bam file in S2, that is, two bases located in read1 and read2 that are aligned to the same position of the reference genome, and mark the overlapping bases that are not paired and do not match the bases in the consensus sequence of the reference genome as sequencing error bases, also called as bases that are incorrectly identified by the sequencer; mark the overlapping bases that are paired and do not match the bases in the consensus sequence of the reference genome as non-sequencing error bases, also called as bases that are incorrectly identified by the non-sequencing instrument, which may be caused by mismatches introduced before sequencing, such as sampling and library construction, biological somatic mutations, etc.; extract the sequencing features of the bases in the overlapping region, including the read direction (i.e., located in read1 or read2), the number of sequencing cycles (cycle), the dinucleotide composed of the previous base in the sequencing amplification direction, and the report quality score (RQS) reported by the instrument;
S4: According to the sequencing features of the bases extracted in S3, the extracted overlapping bases are divided into different groups to form feature bins, that is, the bases with the same sequencing features are divided into a group to generate a feature bin; if the number of bases within the feature bin is less than 30,000, a window is moved up and down along the cycle with a step size of 1 until the number of bases in the feature bin is greater than or equal to 30,000; the base quality score Q is an integer mapping of the probability of base recognition error, that is, Q=−10×1g(P), where P is the probability of base recognition error. When Q=40, P=0.01%. Therefore, at least 10,000 bases must be counted to calculate accurately the bases of Q=40. In this application, 30,000 is set so that there is sufficient data to make the calculated empirical quality value more accurate;
S5: Count the number of sequencing error bases and total bases in each feature bin in S4, and calculate the empirical quality score (EQS) of each bin according to Formula (I):

$$\{ \begin{matrix}empirical quality = - 10 * log 10 ( \frac{mismatches + 1}{bases + 2} ) & ( emperical quality <= 40 ) \\ empirical quality = 40 & ( emperical quality > 40 )\end{matrix} \qquad Formula ( I )$$

In Formula (I), empirical quality represents the empirical quality score (EQS) of the bin, mismatches represent the number of sequencing error bases in the bin, and bases represent the number of all bases in the bin;
S6: A locally weighted regression model (lowess) was used to perform polynomial fitting modeling on the report quality score (RQS) and empirical quality score (EQS) reported by the instrument. The base quality scores reported by the instrument were corrected based on the model to obtain the corrected base quality scores.

Furthermore, in the above S1, the original double-end sequencing data is the sequencing data of the FASTQ file.

Furthermore, in the above S1, removing low-quality sequences includes: removing reads with a length less than 51 bp; sliding the window from the front to the back of the read with a window size of 1 base, and if the average base quality score in the window is ≤1, then filtering out the bases on the right side of the window.

Furthermore, in the above S1, the adapter sequences and low-quality sequences are removed by using the adapter removal software Trimmomatic to obtain a pre-processed FASTQ file.

Furthermore, in the above S1, aligning to the reference genome to generate a Bam file includes aligning the preprocessed FASTQ file to the hg19 human reference genome sequence and generating a Bam file.

Furthermore, in the above S1, the Bam file is generated by aligning to the reference genome using the genome alignment tool BWA mem-M.

Furthermore, in the above S2, the consensus sequence of the reference genome is constructed by taking the base with a mutation frequency ≥99% as the true base type of the site.

Furthermore, in the above S2, reads-level filtering comprises: filtering out read pairs with only one read aligned to the reference genome, read pairs that are not aligned to the reference genome, reads that are non-primary alignments, reads that fail in the platform/vendor quality check, and reads with embedded alignments but not primary representative alignments.

Furthermore, in the above S6, the local weighted regression model is constructed using the lowess method of the sm.nonparametric module of Python, and the parameter frac is set to 0.1.

As the second aspect of the current application, it provides a base quality score correction device for sequencing platform characteristics, comprising:
Data input module: this is used to input raw double-end sequencing data, such as raw fastq sequencing data;
Trim module: this is used to remove the adapter sequence in the original double-end sequencing data to prevent the adapter sequence from affecting the subsequent extraction of bases and distinguish the source of base errors; this module may include commercial software, such as fastp software, to remove the adapter from the input original double-end sequencing data;
Mapping module: this is used to map the input sequencing data without adapters to the reference genome and generate a Bam file mapped to the reference genome; this module may include commercial software, such as samtools view, to map the sequencing data without adapters to the reference genome;
Filtering module: this is used to construct the consensus sequence of the reference genome and perform reads-level filtering for the input Bam file generated and aligned to the reference genome, to construct the consensus sequence of the reference genome, that is, to use the bases with mutation frequencies greater than or equal to 99% as the true base types of the site; reads-level filtering includes filtering out read pairs with only one read aligned to the reference genome, read pairs not aligned to the reference genome, reads with non-primary alignments, reads that failed the platform/supplier quality check, and reads with embedded alignments but not primary representative alignments, but do not includes filtering out the bases in the overlapping parts of read1 and read2 based on the base quality scores, that is, retaining the bases in the overlapping parts of read1 and read2; this module may include commercial software, such as samtools software and python's pysam module, to filter the aligned files output by the mapping module, filter out read pairs with only one read aligned (SAM markers are 4 or 8), and filter out reads with non-primary alignments (not primary alignment, supplementary alignments, SAM markers are 256 and 2048)), filtering out reads that failed the platform or vendor quality check (SAM marker 512);
Overlapping base extraction and error source distinction module (overlapping module): this is used to extract all overlapping bases of read1 and read2 from the filtered Bam file, distinguish the error source according to the coordinate position on the same consensus sequence of the reference genome, whether the bases of read1 and read2 are complementary and paired, and whether they match the bases in the consensus sequence of the reference genome. The bases in read1 that are not paired with the bases of read2 and do not match the consensus sequence of the reference genome are marked as bases derived from sequencing errors; the bases in read1 that are paired with the bases of read2 and do not match the consensus sequence of the reference genome are marked as bases derived from non-sequencing errors. This module may include commercial software, such as Python's pysam module, for overlapping base extraction and error source distinction;
Error rate statistics module (feature_bin module): this is used to extract the features of overlapping bases, including read direction (i.e., located in read1 or read2), sequencing cycle number (cycle), dinucleotide composed of the previous base in the sequencing amplification direction, and base quality score reported by the instrument (report quality score, RQS), and group the bases (bin) according to the features, and count the base error rate in each bin; based on the sequencing error bases in the bin, calculate the empirical quality score (EQS) according to Formula (I);

$$\{ \begin{matrix}empirical quality = - 10 * log 10 ( \frac{mismatches + 1}{bases + 2} ) & ( emperical quality <= 40 ) \\ empirical quality = 40 & ( emperical quality > 40 )\end{matrix} \qquad Formula ( I )$$

In Formula (I), empirical quality represents the empirical quality score (EQS) of the bin, mismatches represent the number of sequencing error bases in the bin, and bases represent the number of all bases in the bin;

Correction model modeling module (modeling module): this is used to fit and model the base quality score (RQS) and empirical quality value (EQS) reported by the instrument under different features using locally weighted regression (lowess) according to the empirical quality value calculated by the error rate statistics module (feature_bin module);

Correction module: this is used to correct the base quality scores reported by the instrument according to the model established by the correction model modeling module, and output the corrected base quality scores.

As the third aspect of the current application, it provides an electronic device, comprising: one or more processors; a storage device on which one or more programs are stored, and when the one or more programs are executed by the one or more processors, the one or more processors implement any of the base quality score correction methods for sequencing platform characteristics of the first aspect mentioned above.

As the fourth aspect of the current application, it provides a computer storage medium having a computer program stored thereon, wherein when the program is executed by a processor, any of the base quality score correction methods for sequencing platform characteristics of the first aspect described above is implemented.

### 3. Beneficial Effects

Compared with the prior art, the current application has the following beneficial effects:
(1) The current application provides a method for correcting base quality scores for sequencing platform characteristics, which uses the bases in the overlapping part of read1 and read2 to distinguish between sequencing error bases and non-sequencing error bases, and can more accurately describe sequencing errors and reflect the systematic preference of the sequencer and the differences in sequencing batches; in addition, the sequencing errors are analyzed and counted under different sequencing features, including those located in read1 or read2, cycle, dinucleotide features and base quality scores reported by the sequencer, and these are used as modeling objects to establish a correction model for correcting base quality scores, avoiding the sequencing errors used for correction in the prior art, which may include sampling and library construction and sample-specific somatic mutations, etc., and making up for the defects of BQSR for cfDNA data, especially for high tumor mutation burden (TMB) samples, and can more accurately reflect the preference of the sequencer and comprehensively improve the credibility of the base quality scores.
(2) The current application provides a method for correcting base quality scores based on the sequencing platform characteristics, which overcomes the reliance of existing correction methods on species mutation databases and the limitations of their scope of application. The current application does not need to rely on a known species mutation database and is applicable not only to sequencing samples for germline mutation analysis but also to sequencing samples for systemic mutations. The current application is not affected by the number of mutations in the samples and is applicable to tumor samples with high and low TMB, thus having a wider range of applicability.
(3) The current application provides a method for correcting base quality scores based on the sequencing platform characteristics, which can clearly distinguish between sequencing errors and non-sequencing errors, accurately measure sequencing errors, and provide a reliable method for improving data detection sensitivity and accuracy.
(4) The current application provides a method for correcting base quality scores based on the sequencing platform characteristics. The method uses overlapping bases to accurately reflect the systematic preferences of sequencing batches and sequencers. Therefore, different samples in the same sequencing batch can share a correction model without the need to construct a sample-specific correction model. This method can greatly improve the efficiency of correcting base quality.

### BRIEF DESCRIPTION OF THE DRAWINGS

FIG. 1 is a distribution diagram of sequencing error rates of the NovaSeq 6000 sequencing platform under different dinucleotides, where the horizontal axis is the mutation type, the vertical axis is the feature type, and the color represents the sequencing error rate value.

FIG. 2 is a distribution diagram of sequencing error rates of the NovaSeq 6000 sequencing platform under different reads and cycles, where the horizontal axis is the mutation type, the vertical axis is the feature type, and the color represents the sequencing error rate value.

FIG. 3 is a distribution diagram of the non-sequencing error rate of the NovaSeq 6000 sequencing platform under different dinucleotides, where the horizontal axis is the mutation type, the vertical axis is the feature type, and the color represents the non-sequencing error rate value.

FIG. 4 is a distribution diagram of the non-sequencing error rate of the NovaSeq 6000 sequencing platform under different reads and cycles, where the horizontal axis is the mutation type, the vertical axis is the feature type, and the color represents the non-sequencing error rate value.

FIG. 5 is a distribution diagram of the accuracy of base quality scores of the NovaSeq 6000 sequencing platform under different dinucleotides and cycles, where the horizontal axis is the feature type and the vertical axis is the accuracy measure of the base quality score, that is the empirical quality value minus the base quality score reported by the instrument.

FIG. 6 shows the relationship between the base quality score and the empirical quality value before and after correction of the NovaSeq 6000 sequencing platform, where Report is the base quality score before correction and Predict is the base quality score after correction.

FIG. 7 is a distribution diagram of the accuracy of the corrected base quality scores of the NovaSeq 6000 sequencing platform under different dinucleotides and cycles, where the horizontal axis is the feature type and the vertical axis is the accuracy measure of the base quality score, i.e., the empirical quality value minus the corrected base quality score.

FIG. 8 is a RMSE distribution diagram of the base quality scores of two sequencing batches (run 1 and run 2) before and after correction on three sequencing platforms: NovaSeq 6000, MGISEQ-2000, and MGISEQ-T7.

FIG. 9 is a distribution diagram of sequencing error rates before and after correction at different dinucleotides using the NovaSeq 6000 sequencing platform with Q30 as the threshold, where the horizontal axis represents mutation type, the vertical axis represents feature type, and the color represents the sequencing error rate value. Report quality represents the base quality score before correction, and Predict quality represents the base quality score after correction.

FIG. 10 is a distribution diagram of sequencing error rates before and after correction under different reads and cycles using the NovaSeq 6000 sequencing platform with Q30 as the threshold, where the horizontal axis is the feature type, the vertical axis is the mutation type, and the color represents the sequencing error rate value. Report quality is the base quality score before correction, and Predict quality is the base quality score after correction.

FIG. 11 is a distribution diagram of the non-sequencing error rate before and after correction at different dinucleotides using the NovaSeq 6000 sequencing platform with Q30 as the threshold, wherein the horizontal axis represents the mutation type, the vertical axis represents the feature type, the color represents the non-sequencing error rate value, wherein Report quality represents the base quality score before correction, and Predict quality represents the base quality score after correction.

FIG. 12 is a distribution diagram of the non-sequencing error rate before and after correction under different reads and cycles using the NovaSeq 6000 sequencing platform with Q30 as the threshold, where the horizontal axis is the feature type, the vertical axis is the mutation type, the color represents the non-sequencing error rate value, and Report quality is the base quality score before correction, and Predict quality is the base quality score after correction.

FIG. 13 is a distribution diagram of sequencing error rates of the MGISEQ-2000 sequencing platform under different dinucleotides, wherein the horizontal axis is the mutation type, the vertical axis is the feature type, and the color represents the sequencing error rate value.

FIG. 14 is a distribution diagram of sequencing error rates of the MGISEQ-2000 sequencing platform under different reads and cycles, where the horizontal axis is the mutation type, the vertical axis is the feature type, and the color represents the sequencing error rate value.

FIG. 15 is a distribution diagram of non-sequencing error rates of the MGISEQ-2000 sequencing platform under different dinucleotides, wherein the horizontal axis is the mutation type, the vertical axis is the feature type, and the color represents the non-sequencing error rate value.

FIG. 16 is a distribution diagram of non-sequencing error rates of the MGISEQ-2000 sequencing platform under different reads and cycles, where the horizontal axis is the mutation type, the vertical axis is the feature type, and the color represents the non-sequencing error rate value.

FIG. 17 is a distribution diagram of the accuracy of base quality scores of the MGISEQ-2000 sequencing platform under different dinucleotides and cycles, wherein the horizontal axis is the feature type and the vertical axis is the accuracy measure of the base quality score, i.e., the empirical quality value minus the instrument-reported base quality score.

FIG. 18 is a diagram showing the relationship between the base quality scores and the empirical quality values before and after correction on the MGISEQ-2000 sequencing platform, where Report is the base quality score before correction and Predict is the base quality score after correction.

FIG. 19 is a distribution diagram of the accuracy of the corrected base quality scores of the MGISEQ-2000 sequencing platform under different dinucleotides and cycles, wherein the horizontal axis is the feature type and the vertical axis is the accuracy measure of the base quality score, i.e., the empirical quality value—the corrected base quality score.

FIG. 20 is a distribution diagram of sequencing error rates before and after correction under different dinucleotides using the MGISEQ-2000 sequencing platform with Q30 as the threshold, wherein the horizontal axis represents mutation type, the vertical axis represents feature type, and the color represents the sequencing error rate value, wherein Report quality represents the base quality score before correction, and Predict quality represents the base quality score after correction.

FIG. 21 is a distribution diagram of sequencing error rates before and after correction under different reads and cycles using the MGISEQ-2000 sequencing platform with Q30 as the threshold, wherein the horizontal axis represents the feature type, the vertical axis represents the mutation type, the color represents the sequencing error rate value, wherein Report quality represents the base quality score before correction, and Predict quality represents the base quality score after correction.

FIG. 22 is a distribution diagram of the non-sequencing error rate before and after correction under different dinucleotides using the MGISEQ-2000 sequencing platform with Q30 as the threshold, wherein the horizontal axis represents the mutation type, the vertical axis represents the feature type, the color represents the non-sequencing error rate value, wherein Report quality represents the base quality score before correction, and Predict quality represents the base quality score after correction.

FIG. 23 is a distribution diagram of the non-sequencing error rate before and after correction under different reads and cycles using the MGISEQ-2000 sequencing platform with Q30 as the threshold, wherein the horizontal axis represents the feature type, the vertical axis represents the mutation type, the color represents the non-sequencing error rate value, wherein Report quality represents the base quality score before correction, and Predict quality represents the base quality score after correction.

FIG. 24 is a distribution diagram of sequencing error rates of the MGISEQ-T7 sequencing platform under different dinucleotides, wherein the horizontal axis is the mutation type, the vertical axis is the feature type, and the color represents the sequencing error rate value.

FIG. 25 is a distribution diagram of sequencing error rates of the MGISEQ-T7 sequencing platform under different reads and cycles, where the horizontal axis is the mutation type, the vertical axis is the feature type, and the color represents the sequencing error rate value.

FIG. 26 is a distribution diagram of non-sequencing error rates of the MGISEQ-T7 sequencing platform under different dinucleotides, wherein the horizontal axis is the mutation type, the vertical axis is the feature type, and the color represents the non-sequencing error rate value.

FIG. 27 is a distribution diagram of non-sequencing error rates of the MGISEQ-T7 sequencing platform under different reads and cycles, where the horizontal axis is the mutation type, the vertical axis is the feature type, and the color represents the non-sequencing error rate value.

FIG. 28 is a distribution diagram of the accuracy of base quality scores of the MGISEQ-T7 sequencing platform under different dinucleotides and cycles, wherein the horizontal axis is the feature type and the vertical axis is the accuracy measure of the base quality score, i.e., the empirical quality value minus the instrument-reported base quality score.

FIG. 29 is the relationship between the base quality score and the empirical quality value before and after correction on the MGISEQ-T7 sequencing platform, where Report is the base quality score before correction, and Predict is the base quality score after correction.

FIG. 30 is a distribution diagram of the accuracy of the corrected base quality scores of the MGISEQ-T7 sequencing platform under different dinucleotides and cycles, wherein the horizontal axis is the feature type and the vertical axis is the accuracy measure of the base quality score, i.e., the empirical quality value—the corrected base quality score.

FIG. 31 is a distribution diagram of sequencing error rates before and after correction under different dinucleotides using the MGISEQ-T7 sequencing platform with Q30 as the threshold, wherein the horizontal axis represents mutation type, the vertical axis represents feature type, and the color represents the sequencing error rate value, wherein Report quality represents the base quality score before correction, and Predict quality represents the base quality score after correction.

FIG. 32 is a distribution diagram of sequencing error rates before and after correction under different reads and cycles using the MGISEQ-T7 sequencing platform with Q30 as the threshold, wherein the horizontal axis represents the feature type, the vertical axis represents the mutation type, the color represents the sequencing error rate value, wherein Report quality represents the base quality score before correction, and Predict quality represents the base quality score after correction.

FIG. 33 is a distribution diagram of the non-sequencing error rate before and after correction under different dinucleotides using the MGISEQ-T7 sequencing platform with Q30 as the threshold, wherein the horizontal axis represents the mutation type, the vertical axis represents the feature type, the color represents the non-sequencing error rate value, wherein Report quality represents the base quality score before correction, and Predict quality represents the base quality score after correction.

FIG. 34 is a distribution diagram of the non-sequencing error rate before and after correction under different reads and cycles using the MGISEQ-T7 sequencing platform with Q30 as the threshold, wherein the horizontal axis represents the feature type, the vertical axis represents the mutation type, the color represents the non-sequencing error rate value, wherein Report quality represents the base quality score before correction, and Predict quality represents the base quality score after correction.

### DETAILED DESCRIPTION OF THE EMBODIMENTS

The current application is further described below in conjunction with specific examples.

It should be noted that the terms such as “upper”, “lower”, “left”, “right”, “middle” etc. cited in this specification are only for the convenience of description and are not used to limit the scope of implementation. Changes or adjustments to their relative relationships should be regarded as the scope of implementation of the present invention without substantially changing the technical content.

Unless otherwise defined, all technical and scientific terms used herein have the same meaning as commonly understood by one of ordinary skill in the art to which the invention belongs; the term “and/or” used herein includes any and all combinations of one or more of the associated listed items.

If no specific conditions were specified in the examples, the experiments were carried out according to conventional conditions or conditions recommended by the manufacturer. The reagents or instruments used without indicating the manufacturer are all conventional products that can be purchased from the market.

As used herein, the term “about” is used to provide flexibility and imprecision associated with a given term, measurement, or value. The degree of flexibility for a particular variable can be readily determined by one skilled in the art.

As used herein, the term “at least one of” is intended to be synonymous with “one or more of.” For example, “at least one of A, B, and C” explicitly includes only A, only B, only C, and combinations of each thereof.

Concentrations, amounts, and other numerical data may be presented herein in a range format. It should be understood that such range format is used merely for convenience and brevity and should be interpreted flexibly to include not only the values expressly recited as the limits of the range, but also to include all individual values or sub-ranges within the range, as if each value and sub-range were expressly recited. For example, a numerical range of about 1 to about 4.5 should be interpreted to include not only the explicitly recited limit of 1 to about 4.5, but also include individual numbers (such as 2, 3, 4) and sub-ranges (such as 1 to 3, 2 to 4, etc.). The same principle applies to ranges reciting only one numerical value, such as “less than about 4.5”, which should be interpreted to include all the aforementioned values and ranges. Furthermore, this interpretation should apply regardless of the breadth of the scope or features being described.

The original sequencing data FASTQ files of this application are derived from three sequencing platforms, NovaSeq 6000, MGISEQ-2000, and MGISEQ-T7, respectively. Specifically, the MagMAX™ Free DNA Isolation Kit (ThermoFisher) was used to extract cfDNA from plasma samples; the KAPA HyperPrep Library Construction Kit (Roche) was used to construct a plasma library; the Panel_P probe (IDT synthesis) was used to enrich the target region of the plasma library, where the Panel_P probe covers a 2.1 Mb interval of the genome, covering a total of 769 genes in 207 important pathways related to the occurrence and development of tumors; and the original sequencing data FASTQ files were obtained by sequencing using three sequencing platforms, NovaSeq 6000, MGISEQ-2000 and MGISEQ-T7.

### Example 1

This example provides a method for correcting base quality scores of the NovaSeq 6000 sequencing platform, which specifically includes the following steps:

S1: The raw sequencing data FASTQ file is preprocessed and aligned to the reference genome to generate a Bam file. The preprocessing includes removing adapter sequences and low-quality bases. Removing low-quality bases includes: 1) removing reads with a length less than 51; 2) sliding a window from the front to the back of the read with a window size of 1 base. If the average base quality score in the window is less than or equal to 1, the bases on the right side of the window are filtered out. In the example, the removal of reads with a length less than 51 is completed by a module of commercial software, such as calling fastp 0.20.0 and using—length_required 51 to remove. In the example, a sliding window is performed from the front to the back of the read with a window size of 1 base. If the average base quality score in the window is less than or equal to 1, the bases on the right side of the window are filtered out by a module of commercial software, such as calling fastp 0.20.0 and using—cut_right-W1-M1 to remove. In an example, the removal of the adapter sequence is performed by a module of a commercial software, such as adapter_fasta TruSeq3-PE.fa. In an example, the aligning to a reference genome to generate a Bam file comprises aligning the preprocessed data to the hg19 human reference genome sequence and generating a Bam file. In an example, the alignment to the reference genome to generate a Bam file is completed by a module of commercial software, such as calling the BWA-MEM algorithm of bwa-0.7.17 to align the reads of double-end sequencing to the hg19 human reference genome sequence, using the view of samtools-1.3 to convert the aligned sam file into a bam file, using the sort of samtools-1.3 to sort, and indexing the sorted bam file.

S2: Using the Bam file generated in S1 as the input file, construct the consensus sequence of the reference genome and perform reads-level filtering, where: construct the consensus sequence of the reference genome, i.e., take the bases with mutation frequency greater than or equal to 99% as the true base type of the site; reads-level filtering includes filtering out read pairs that are not aligned to the reference genome, reads that are not the main alignment, reads that fail the platform/supplier quality check, and reads that are embedded in the alignment but not the main representative alignment. At the same time, the bases in the overlapping parts of read1 and read2 are not filtered according to the base quality score. In the examples, reads with non-primary alignments, reads that failed platform/vendor quality checks, and reads that align in pairs but not with the primary representative alignment are commonly defined when filtered by commercial software. In the example, reads-level filtering is completed by a module of commercial software, which sequentially sets flag_filter to 2820, read unmapped, flag (4,8), not primary alignment, flag 256, read fails platform/vendor quality checks, flag 512, supplementary alignment, flag 2048. In the example, the min_base_quality parameter is set to 0, and ignore_overlaps is set to false, that is, the bases in the overlapping parts of read1 and read2 are not filtered according to the base quality scores, but both are retained for subsequent analysis. In an example, reads information is extracted by commercial software. In the example, the pileup method of the pysam module of Python 3.9.12 is used to read the reads information aligned at the position from the bam file site by site according to the position of the reference genome.

S3: Extract overlapping bases and their characteristic information from the filtered Bam file, and distinguish the sources of mismatch errors, including: based on the filtering results of S2, extract the reads pairs aligned to a certain position in the genome according to the read name, distinguish whether the source of the reads pairs is F1R2 or F2R1, that is, determine whether it belongs to read1 or read2; extract the base of the read aligned to the site, and the dinucleotide (dinucleotide) composed of the previous base in the sequencing amplification direction; the sequencing cycle number of the base (cycle); extract the base quality score reported by the sequencer(RQS); mark the bases of read1 that do not match the bases of read2 and the bases that do not match the bases in consensus sequence of the reference genome as sequencing error bases; pair the bases of read1 and read2, and the bases that do not match the bases in the consensus sequence of the reference genome are marked as non-sequencing error bases. In the example, for example, read E100034389L1C010R03202285505:TCA_TAT is a sequencing fragment derived from the positive strand of the consensus sequence of the reference genome, so its reads composition is F1R2, that is, read1 is aligned to the positive strand of the reference gene, with a total length of 95 bp, and the 80th base C is aligned to the A site of 101620719 of the positive strand chr10 of the consensus sequence of the reference genome, and the base quality score given by its sequencer is 14, the cycle is 80, and the dinucleotide is TA, while the corresponding read2 has a total length of 95 bp, its base T is aligned to the negative strand of the 101620719 site of the consensus sequence of the reference genome chr10, and the sequencing quality value given by the sequencer is 37, the cycle is 22, and the dinucleotide is CT. Since the original positive strand base of the 101620719 site of the consensus sequence of the reference genome chr10 is A and the negative strand is T, the C base aligned to this position by read1 is marked as a sequencing error, and the T aligned to this position by read2 is marked as the correct sequencing base. Read E100034389L1C003R02404263679:AGC_ATC is a sequenced fragment derived from the negative strand of the consensus sequence of the reference genome, so its reads composition is F2R1, that is, read2 is aligned to the positive strand of the reference gene, with a total length of 95 bp, and the 68th base A is aligned to the 104268962 G site of chr10 of the positive strand of the consensus sequence of the reference genome, and the base quality score given by its sequencer is 38, the cycle is 68, and the dinucleotide is TG, while the corresponding read1 has a total length of 95 bp, its base T is aligned to the C site on the negative strand of the consensus sequence of the reference genome chr10 104268962, and the base quality score given by the sequencer is 36, the cycle is 5, and the dinucleotide is AC. Since the original positive strand base of the consensus sequence of the reference genome chr10 104268962 site is G and the negative strand is C, the T aligned to this position by read1 and the A aligned to this position by read2 are marked as non-sequencing errors.

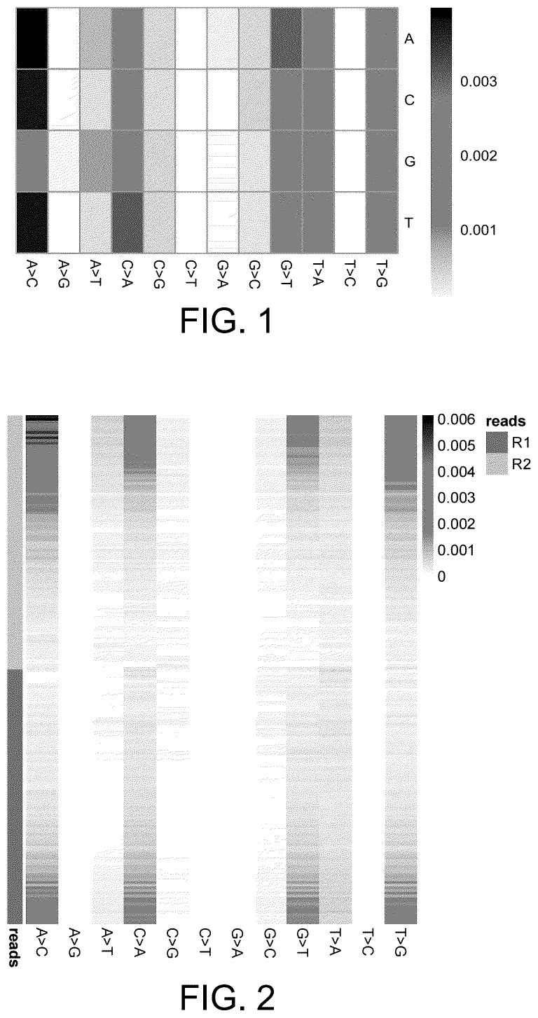

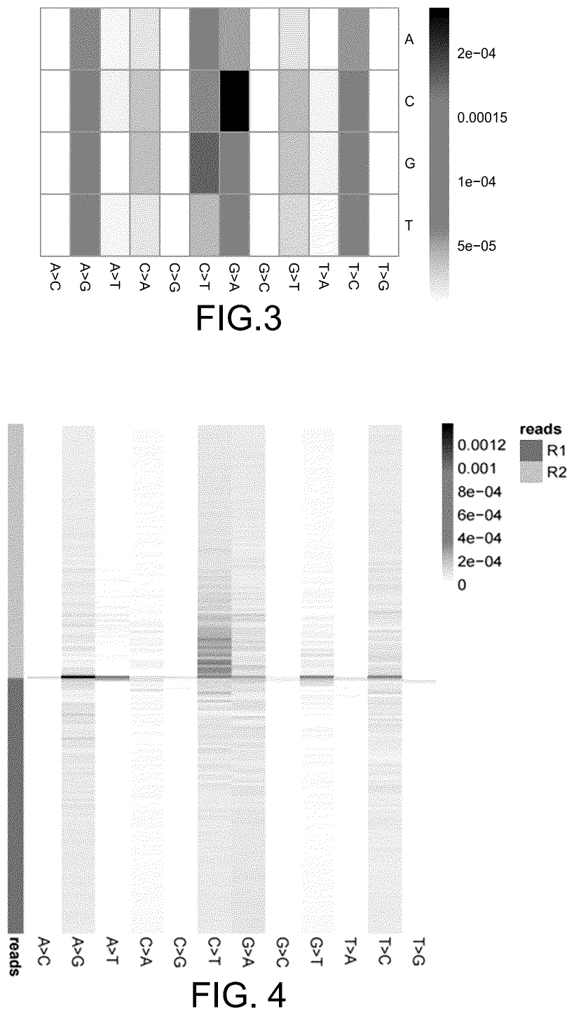

S4: Evaluation of systematic preference of sequencing platform and mismatch pattern caused by non-sequencing, including the following steps: Based on the results of S3, the sequencing error pattern and non-sequencing error pattern of the sequencer are described using read position, dinucleotide and cycle as features, that is, the proportion and distribution of sequencing errors and non-sequencing errors of bases in each cycle of different dinucleotide and different read sources are counted. The results are shown in FIGS. 1 to 4. It can be seen from the figures that the error rates of sequencing and non-sequencing errors are significantly different in different sequencing features (dinucleotide, cycle), such as the A(A) source of sequencing errors. C) and T (A>C) have the highest error rates, and the sequencing error rate increases significantly along the sequencing cycle; the error rates of G (C>T) and C (G>A), which are not sources of sequencing errors, are the highest, and the error rate in the first cycle of Read2 is the highest.

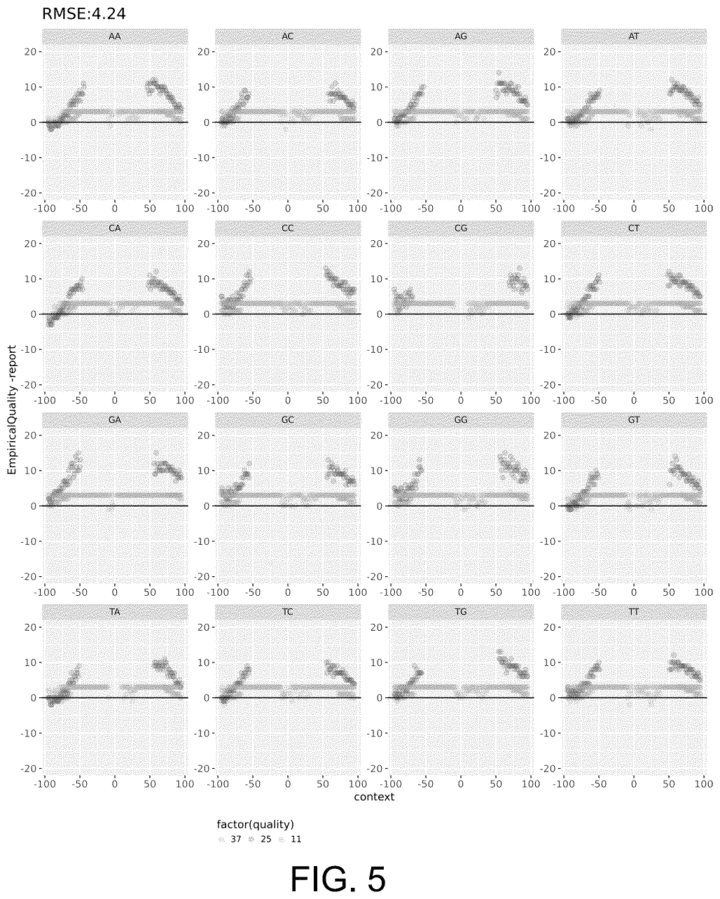

S5: Base quality score accuracy assessment, the base quality score accuracy is the difference between the empirical quality value and the base quality score reported by the sequencer, specifically comprising the following steps: Based on the analysis results of S4, statistically analyze the relationship between the given base quality score and the empirical quality value of the NOVA-seq 6000 sequencing platform. Statistical analysis found that there are differences in the error rates of different sequencing features (read orientation, dinucleotide, cycle). In order to measure the accuracy of the base quality score of the sequencing platform under different sequencing features (read orientation, dinucleotide, cycle), the extracted overlapping bases are divided into different groups to form feature bins, that is, the bases with the same sequencing features are divided into a small group to generate a feature bin. The total number of bases and the number of bases with sequencing errors under each cycle and dinucleotide of different read orientations under the base quality score reported by the sequencer are counted, and the empirical quality value of the base is calculated to measure the accuracy of the base quality score of the sequencing platform. The calculation formula of the empirical quality value is shown in Formula (1). The accuracy distribution diagram of base quality scores of the NovaSeq 6000 sequencing platform under different dinucleotides and cycles is shown in FIG. 5. It can be seen from the figure that there are obvious differences in the accuracy of base quality scores of different sequencing features (dinucleotide, cycle).

$$\{ \begin{matrix}empirical quality = - 10 * log 10 ( \frac{mismatches + 1}{bases + 2} ) & ( emperical quality <= 40 ) \\ empirical quality = 40 & ( emperical quality > 40 )\end{matrix} \qquad Formula ( 1 )$$

Among them, empirical quality represents the empirical quality score (EQS) of the bin, mismatches represents the number of sequencing error bases in the bin, and bases represents the number of all bases in the bin;

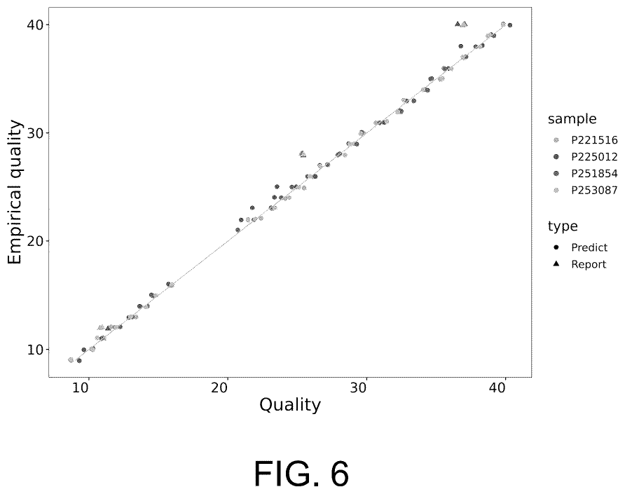

S6: The Lowess method of Python's sm.nonparametric module, i.e., the locally weighted regression model (Lowess), was used to perform polynomial fitting modeling on the report quality score (RQS) and empirical quality score (EQS) reported by the instrument, with the parameter frac set to 0.1. Based on the model, the base quality scores reported by the instrument are corrected to obtain the corrected base quality scores. The distribution of the relationship between the base quality scores before and after correction and the empirical base quality scores is shown in FIG. 6. It can be seen from the figure that compared with the base quality scores reported by the instrument before correction, the quality values after correction are closer to the empirical quality values;

S7: Accuracy assessment of base quality scores of different sequencing batches after correction.

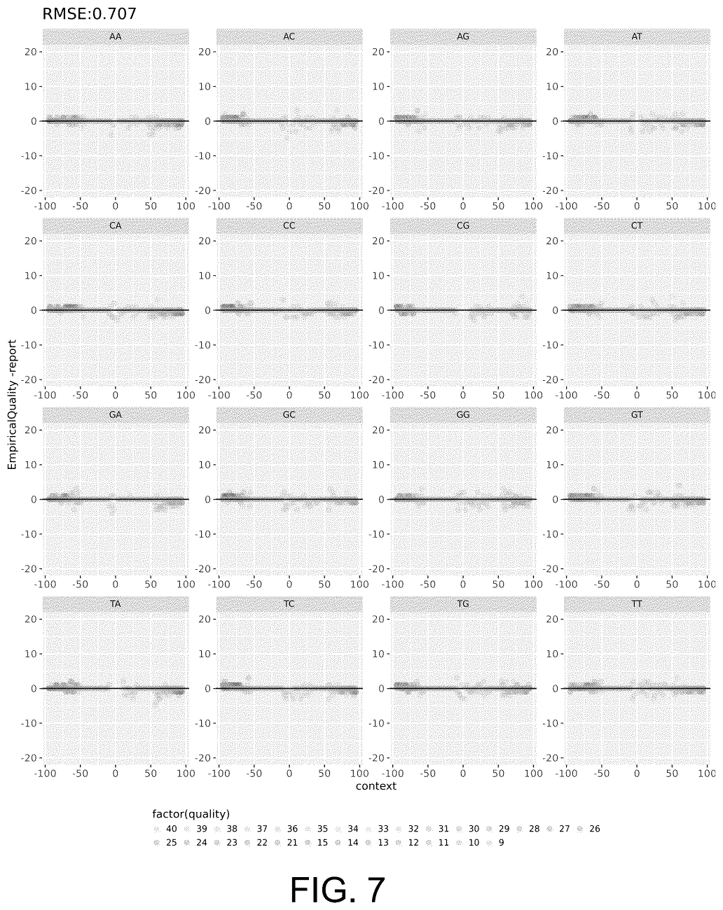

(1) Referring to step S5, the accuracy of the corrected base quality scores is evaluated. The accuracy distribution of the base quality scores of different dinucleotides and cycles after correction is shown in FIG. 7. It can be seen that after the base quality score correction, the deviations of the base quality scores of different dinucleotides and cycles are well corrected.

(2) Formula (2) is used to calculate the root mean square error (RMSE) to measure the accuracy of the base quality scores before and after correction at the sample level. The root mean square error is the square root of the ratio of the square of the deviation between the predicted value and the true value to the number of observations n. It measures the deviation between the predicted value and the true value and is more sensitive to outliers in the data:

$$RMSE = \sqrt{\frac{1}{N} {\sum }_{i = 1}^{n} {( {Y}_{i} - f ( {x}_{i} ) )}^{2}} \qquad Formula ( 2 )$$

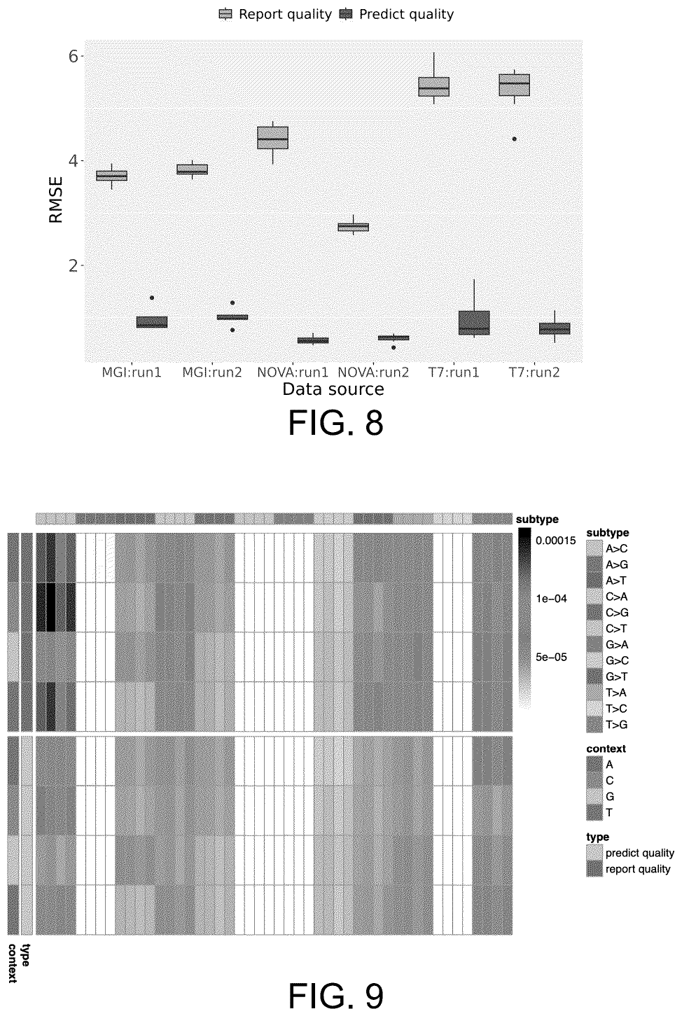

The RMSE distribution between the base quality scores and the empirical quality values before and after correction of two batches of 13 samples (run1:4; run2:9) on the NOVA-seq 6000 sequencing platform is shown in FIG. 8. The corrected base quality scores can more accurately describe the base error rate.

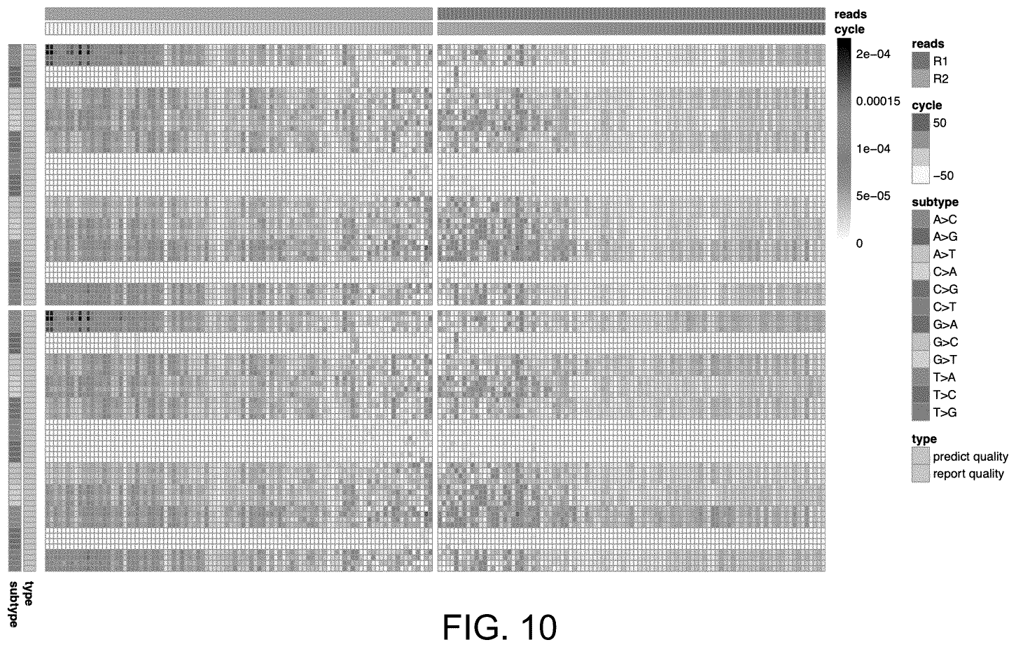

(3) Using Q30 as the threshold, the distribution of sequencing error rates before and after correction was compared. For the four NOVA-seq 6000 samples, the base quality score threshold of 30 before and after correction was used to filter the bases in the overlapping regions, and the sequencing error rates under different dinucleotide types and cycles were recalculated based on the filtering results. As shown in FIGS. 9 and 10, base quality score correction can effectively reduce the sequencing deviation caused by different dinucleotide types and cycles, and effectively improve the uniformity of detection accuracy under the same threshold.

(4) Using Q30 as the threshold, the distribution of non-sequencing error rates before and after correction was compared. For the four NOVA-seq 6000 samples, the base quality score threshold of 30 before and after correction was used, and the non-sequencing error rates under different Dinucleotide types and different cycles were recalculated based on the filtering results.

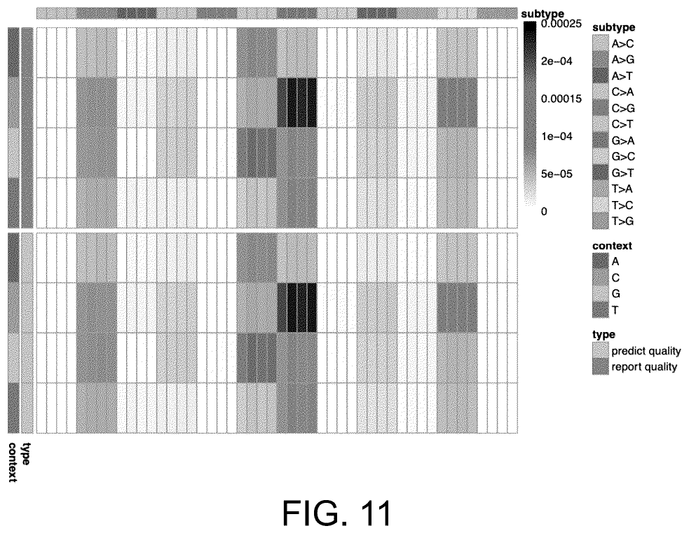

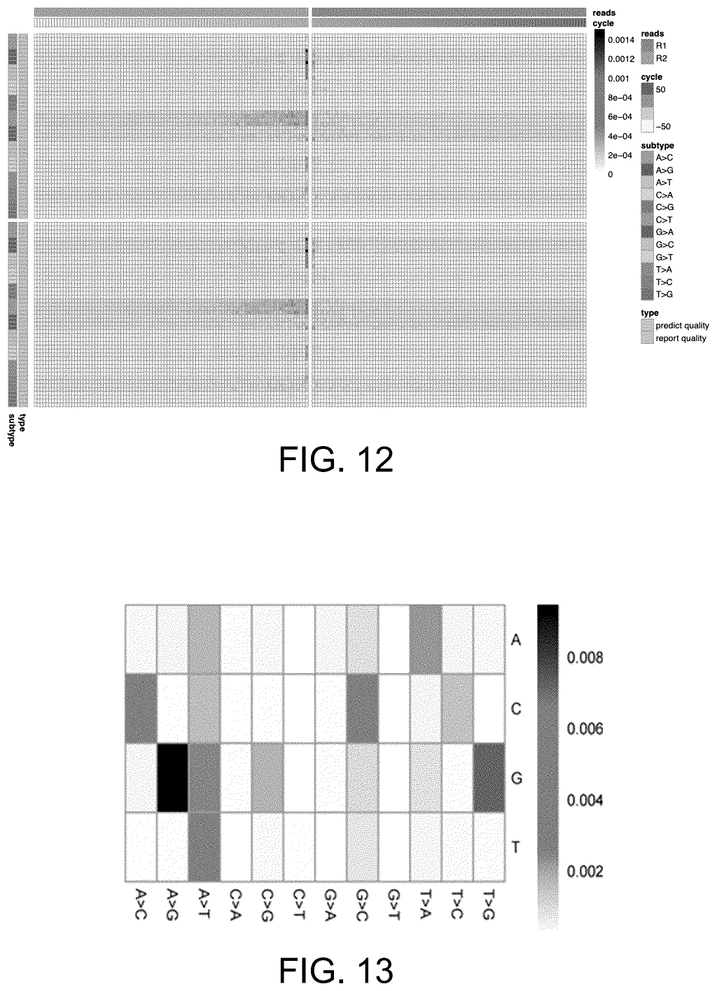

As shown in FIGS. 11 and 12, the non-sequencing error rates before and after correction did not change significantly, which was consistent with expectations.

### Example 2

This example provides a method for correcting base quality scores of the MGISEQ-2000 sequencing platform, and the specific steps are shown in Example 1.

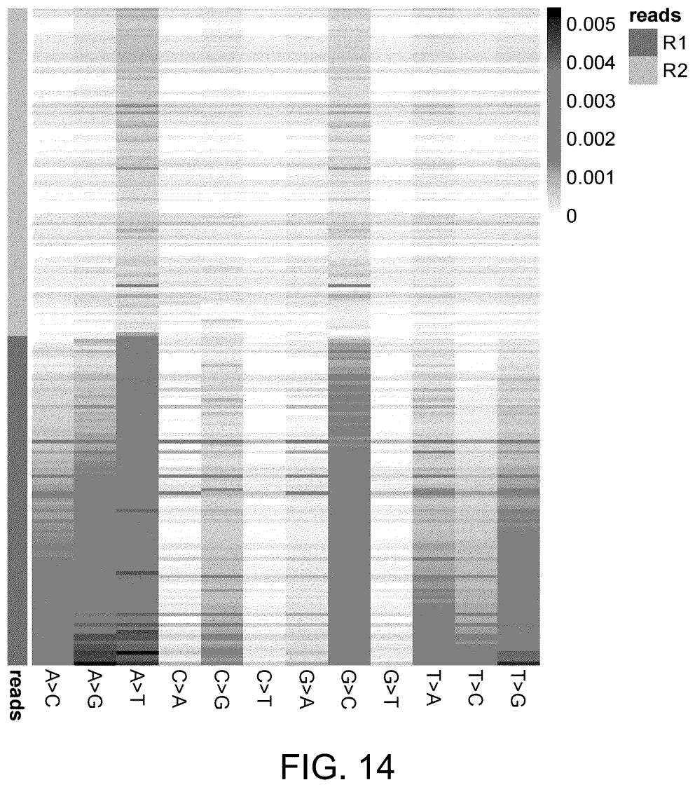

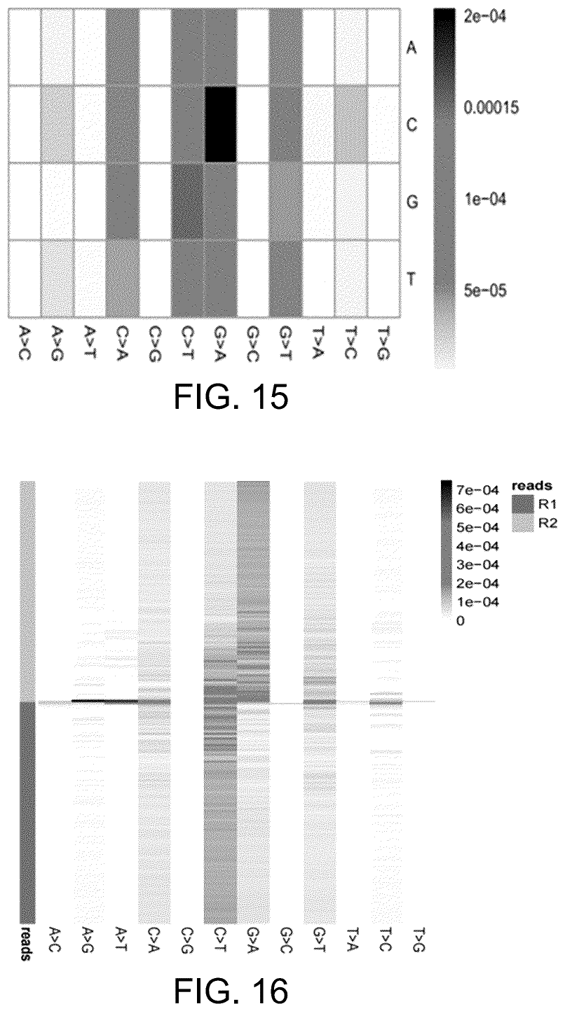

Evaluation of the systematic preference of the sequencing platform and the mismatch pattern caused by non-sequencing, the ratio and distribution of sequencing errors and mismatches caused by non-sequencing in each cycle of different dinucleotides and different read chain sources. The results are shown in FIGS. 13 to 16. It can be seen from the figure that the error rates of sequencing and non-sequencing errors are significantly different in different sequencing features (dinucleotide, cycle). For example, the sequencing error source G(A<G), G (T>G) and C (G>C) were the highest in error rate; the error rates of sequencing along the read1 sequencing cycle did not change significantly, but increased significantly during the read2 sequencing cycle; the error rates of G (C>T) and C (G>A), which were not sources of sequencing errors, were the highest, and the error rate of the first cycle of Read2 was the highest. The results were consistent with the trend of NOVA-seq6000 in the same library construction batch.

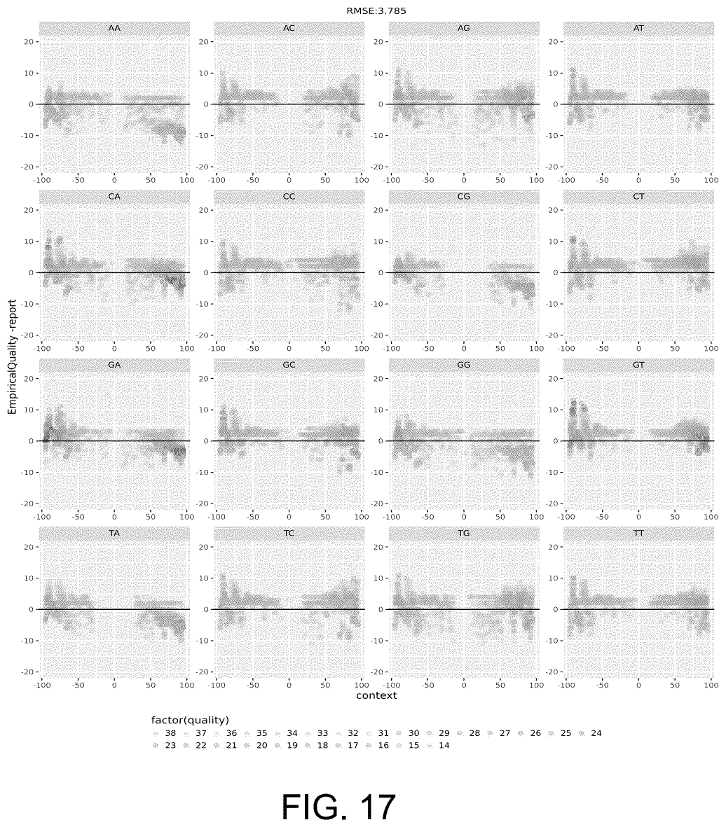

The accuracy distribution of base quality scores of the MGISEQ-2000 sequencing platform under different dinucleotides and cycles is shown in FIG. 17. From the figure, it can be seen that there are obvious differences in the accuracy of base quality scores of different sequencing features (dinucleotide, cycle) of MGISEQ-2000. The accuracy of the quality values of some bases at the end of the reads is higher than the empirical quality value, which underestimates the sequencing error rate under this feature.

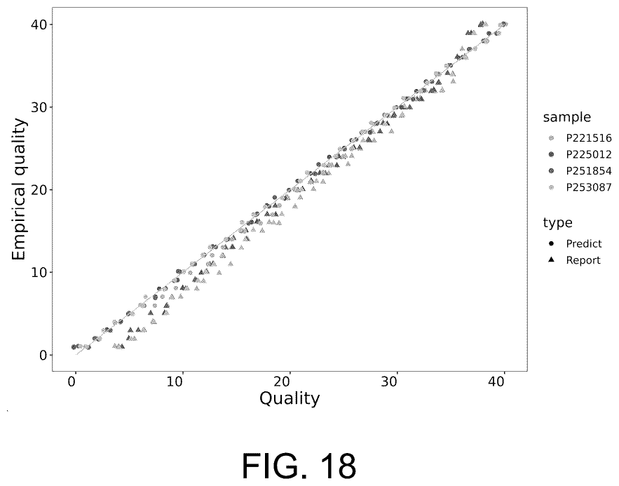

The base quality score correction model was constructed, and the base quality scores reported by the sequencer under different feature bins of the MGISEQ-2000 sequencing platform were fitted with the empirical base quality scores using the local weighted average algorithm, and the base quality scores were corrected. The relationship distribution between the base quality scores and the empirical base quality scores before and after correction is shown in FIG. 18. It can be seen from the figure that compared with the base quality scores given by the instrument before correction, the quality values after correction are closer to the empirical quality values.

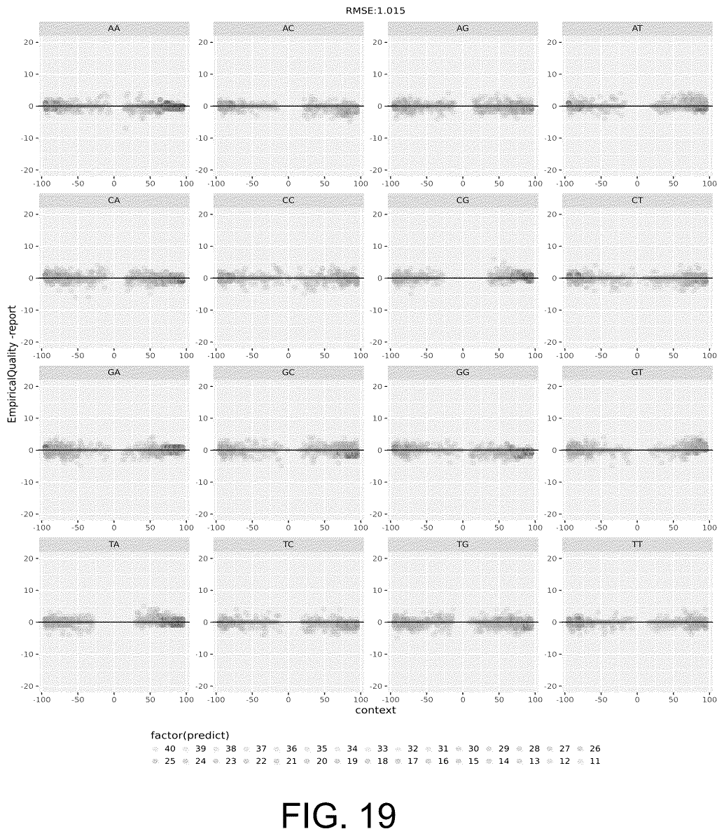

The accuracy distribution of the base quality scores after correction under different dinucleotides and cycles of the MGISEQ-2000 sequencing platform is shown in FIG. 19. It can be seen that the base quality scores given by the sequencing platform are deviated from the empirical quality values, and there are also differences under different dinucleotides and cycles. After the base quality score correction, the deviations are well corrected.

The RMSE distribution between the base quality scores and the empirical quality values before and after correction for a total of 9 samples (run 1:4; run 2:5) of the MGISEQ-2000 sequencing platform is shown in FIG. 8. The corrected base quality scores can more accurately describe the base error rate.

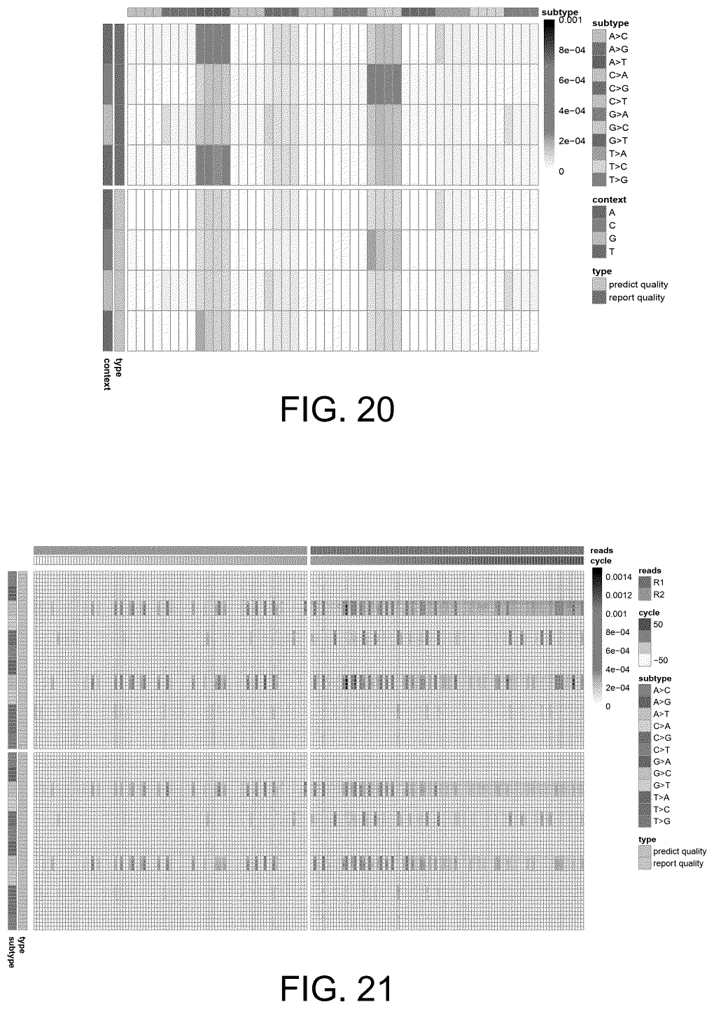

The distribution of sequencing error rates before and after correction was compared using Q30 as the threshold. For the four samples of MGISEQ-2000, the base quality score threshold of 30 before and after correction was used to filter the bases in the overlapping region, and the sequencing error rates under different dinucleotide types and different cycles were recalculated based on the filtering results. As shown in FIGS. 20 and 21, the base quality score correction can effectively reduce the sequencing deviation caused by different dinucleotide types and different cycles, and effectively improve the uniformity of detection accuracy under the same threshold.

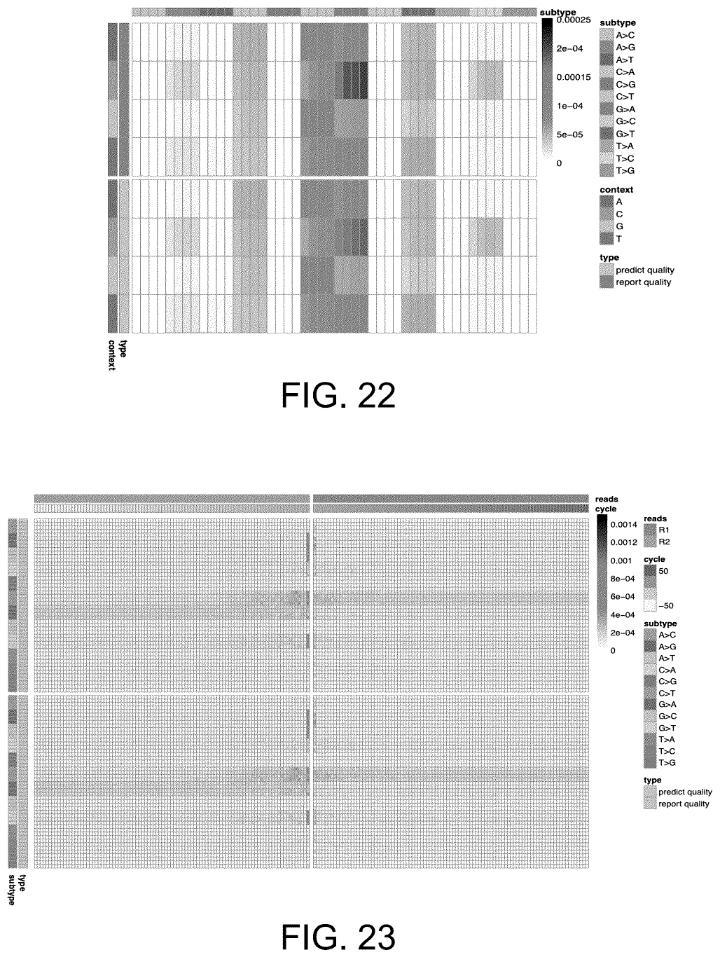

The distribution of non-sequencing error rates before and after correction was compared using Q30 as the threshold. For the four NOVA-seq 6000 samples, the base quality score threshold of 30 before and after correction was used to filter the bases in the overlapping regions, and the non-sequencing error rates under different Dinucleotide types and different cycles were recalculated based on the filtering results. As shown in FIGS. 22 and 23, the non-sequencing error rates before and after correction did not change significantly, which was consistent with expectations.

### Example 3

This example provides a method for correcting base quality scores of the MGISEQ-T7 sequencing platform, and the specific steps are described in Example 1.

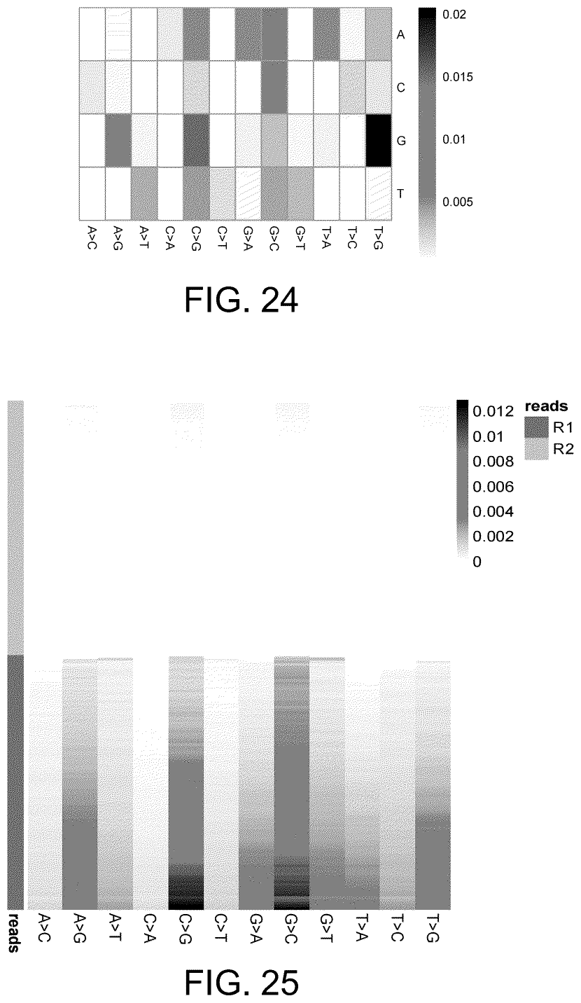

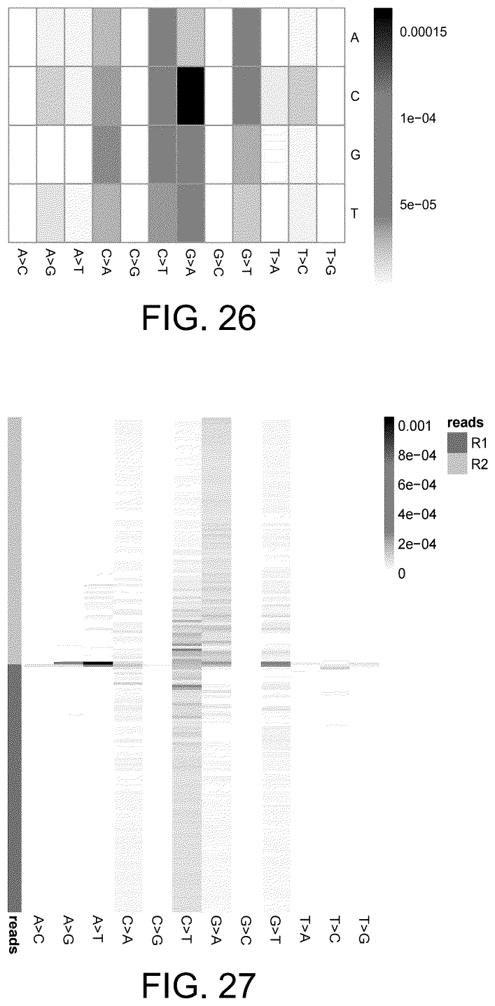

Evaluation of the systematic preference of the sequencing platform and the mismatch pattern caused by non-sequencing, the ratio and distribution of sequencing errors and mismatches caused by non-sequencing in each cycle of different dinucleotides and different read chain sources. The results are shown in FIGS. 24 to 27. It can be seen from the figure that the error rates of sequencing and non-sequencing errors are significantly different in different sequencing features (dinucleotide, cycle), such as G (T<G), G (C>G), G (A>G), and C (G>G), which have the highest error rates in the sources of sequencing errors; The change in sequencing error rate along the read1 sequencing cycle is not significant, but the sequencing error rate significantly increases during the read2 sequencing cycle; the error rates of G(C>T) and C(G>A) which were not sources of sequencing errors were the highest, and the error rate of the first cycle of read2 was the highest. The results were consistent with the trends of MGISEQ-2000 and NOVA-seq6000 in the same library construction batch.

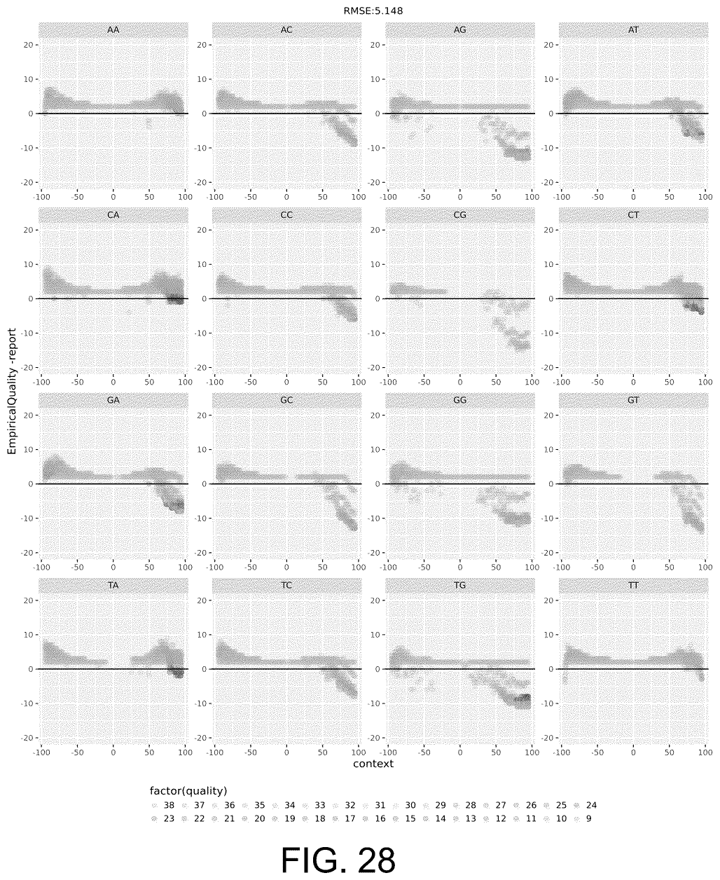

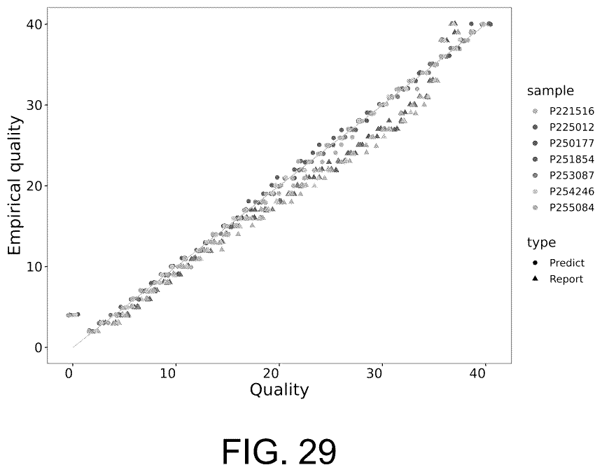

The accuracy distribution diagram of the base quality scores of the MGISEQ-T7 sequencing platform under different dinucleotides and cycles is shown in FIG. 28. It can be seen from the figure that at the end of read2 along the cycle direction, the base quality given by the instrument is much higher than the empirical quality value, that is, the error rate is underestimated.

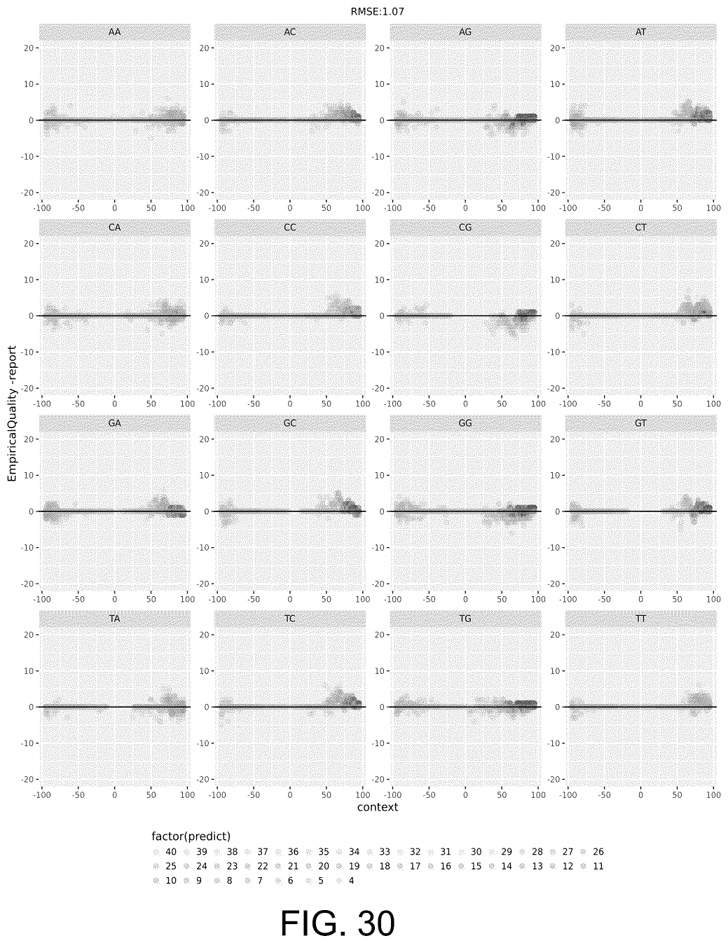

The accuracy distribution of the base quality scores of the MGISEQ-T7 sequencing data after correction under different dinucleotides and cycles is shown in FIG. 30. It can be seen from the figure that the deviation is well corrected after the base quality score correction.

The RMSE distribution between the base quality scores and the empirical quality values before and after correction for a total of 18 samples (run 1: 7; run 2: 11) in two batches of the MGISEQ-T7 sequencing platform is shown in FIG. 10. The corrected base quality scores can more accurately describe the base error rate.

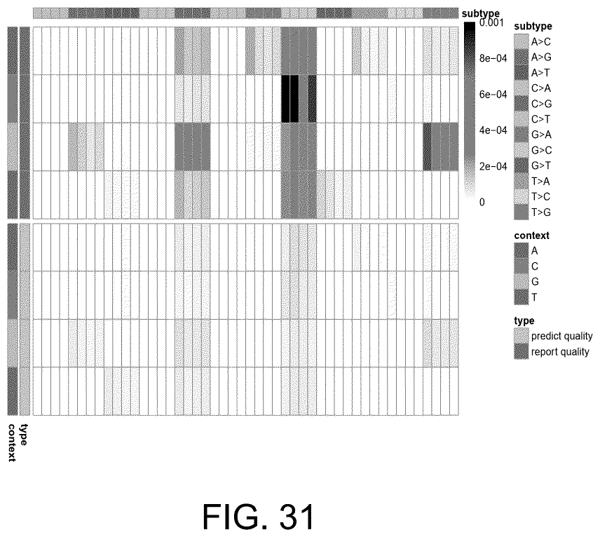

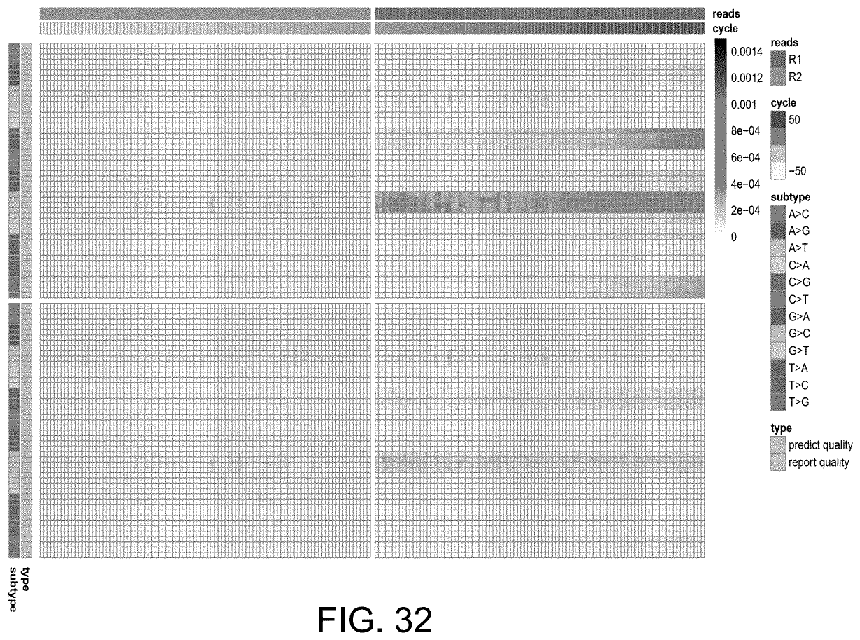

The distribution of sequencing error rates before and after correction was compared using Q30 as the threshold. For the four samples of MGISEQ-T7, the base quality score threshold of 30 before and after correction was used to filter the bases in the overlapping region, and the sequencing error rates under different dinucleotide types and different cycles were recalculated based on the filtering results. As shown in FIGS. 31 and 32, the base quality score correction can effectively reduce the sequencing deviation caused by different dinucleotide types and different cycles, and effectively improve the uniformity of detection accuracy under the same threshold.

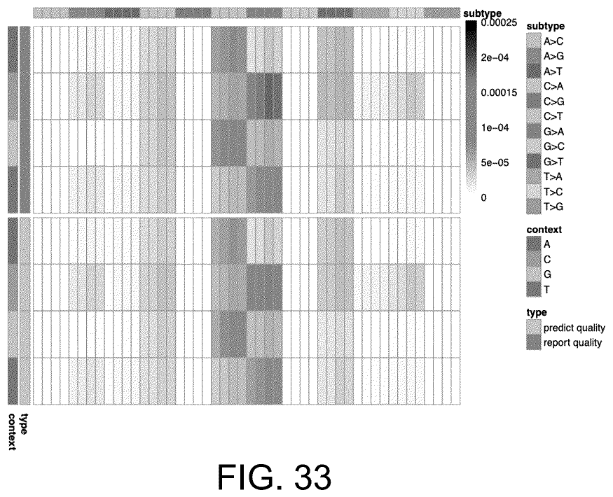

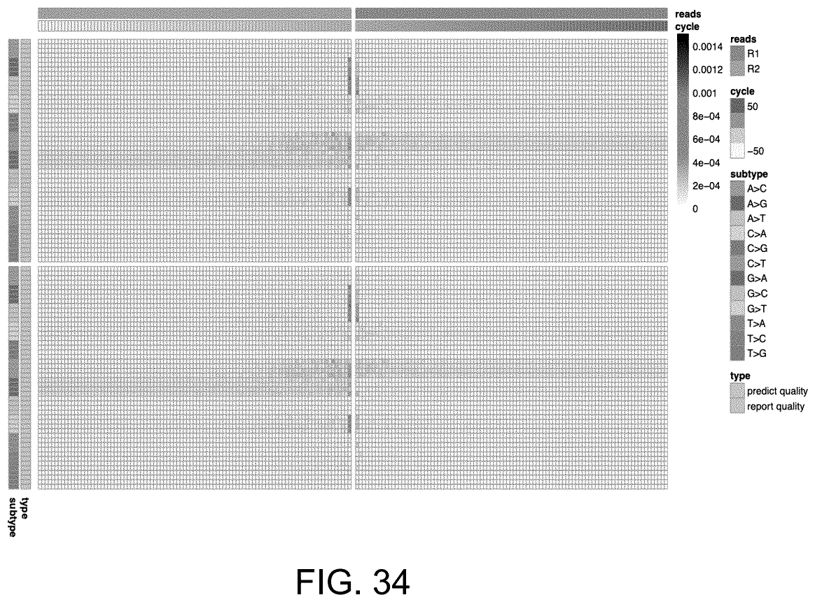

The distribution of non-sequencing error rates before and after correction was compared using Q30 as the threshold. For the four NOVA-seq 6000 samples, the base quality score threshold of 30 before and after correction was used to filter the bases in the overlapping regions, and the non-sequencing error rates under different Dinucleotide types and different cycles were recalculated based on the filtering results. As shown in FIGS. 33 and 34, the non-sequencing error rates before and after correction did not change significantly, which was consistent with expectations.

### Example 4

This example provides a base quality score correction device for sequencing platform characteristics, including the following modules:
Adapter removal module: used to remove adapter sequences from the original double-end sequencing data;
Alignment module: used to align the input sequencing data without adapters to the reference genome and generate a Bam file aligned to the reference genome;
Filtering module: used to construct a consensus sequence of the reference genome for the input Bam file generated and aligned to the reference genome and perform reads-level filtering, wherein the consensus sequence of the reference genome is constructed, that is, the base with a mutation frequency greater than or equal to 99% is used as the true base type of the site; the reads-level filtering includes filtering out read pairs with only one read aligned to the reference genome, read pairs not aligned to the reference genome, reads with non-primary alignments, reads that failed the platform/supplier quality check, and reads with embedded alignments but not primary representative alignments, but the bases in the overlapping part of read1 and read2 are not filtered according to the base quality score, that is, the bases in the overlapping part of read1 and read2 are retained;
Overlapping base extraction and error source distinction module: used to extract all overlapping bases of read1 and read2 from the filtered Bam file, distinguish the error source according to the coordinate position on the same consensus sequence of the reference genome, whether the bases of read1 and read2 are complementary and paired, and whether they match the bases in consensus sequence of the reference genome. The bases of read1 that are not paired with the bases of read2 and the bases that do not match the consensus sequence of the reference genome are marked as bases derived from sequencing errors;
Error rate statistics module: used to extract the characteristics of overlapping bases, including read direction, sequencing cycle number, dinucleotides composed of the previous base in the sequencing amplification direction, and base quality scores reported by the instrument, and divide the bases into different bins according to the characteristics, and count the base error rate in each bin; based on the sequencing error bases in the bin, the empirical quality value is calculated according to Formula (1);

$$\{ \begin{matrix}empirical quality = - 10 * log 10 ( \frac{mismatches + 1}{bases + 2} ) & ( emperical quality <= 40 ) \\ empirical quality = 40 & ( emperical quality > 40 )\end{matrix} \qquad Formula ( 1 )$$

Among them, empirical quality represents the empirical quality value of the bin, mismatches represents the number of sequencing error bases in the bin, and bases represents the number of all bases in the bin;
Correction model building module: It is used to fit and model the base quality scores and empirical quality values reported by the instrument under different characteristics using local weighted regression according to the empirical quality values calculated by the error rate statistics module. The local weighted regression model is built using the lowess method of the sm.nonparametric module of Python, and the parameter frac is set to 0.1.

Correction module: used to correct the base quality scores reported by the instrument according to the model established by the correction model building module, and output the corrected base quality scores.

What is claimed is:

1. A base quality scores correction method for sequencing platform characteristics, comprising the following steps:
S1: preprocessing a raw double-end sequencing data and aligning the preprocessed raw double-end sequencing data to a reference genome to generate a Bam file, the preprocessing includes removing an adapter sequence and low-quality sequences;
S2: using the Bam file generated in S1 as input, building a consensus sequence of the reference genome and performing a reads-level filtering, without filtering bases in overlapping parts of read1 and read2 based on a base quality score;
S3: extracting all the overlapping bases of read pairs from the Bam file filtered in S2, marking the overlapping bases that are not paired and without matching the bases in the consensus sequence of the reference genome as sequencing error bases; extracting sequencing features of the bases in the overlapping parts, including read direction, sequencing cycle number, dinucleotides composed of a previous base in a sequencing amplification direction, and base quality scores reported by an instrument;
S4: according to sequencing features of the overlapping bases extracted in S3, dividing the extracted overlapping bases into different groups to form feature bins; if a number of the bases in the feature bin is less than 30,000, a window is moved up and down along a cycle with a step size of 1 until the number of the bases in the feature bin is greater than or equal to 30,000;
S5: counting a total number of the bases and a number of the bases sequenced incorrectly in each feature bin in S4, and calculating an empirical quality value of each feature bin according to Formula (I):
$$\{ \begin{matrix}empirical quality = - 10 * log 10 ( \frac{mismatches + 1}{bases + 2} ) & ( emperical quality <= 40 ) \\ empirical quality = 40 & ( emperical quality > 40 )\end{matrix} , \qquad Formula ( I )$$
in Formula (I), empirical quality represents the empirical quality value of the feature bin, mismatches represent a number of sequencing error bases in the feature bin, and bases represent a number of all bases in the feature bin; and
S6: using a local weighted regression model to perform a polynomial fitting modeling on the base quality scores and the empirical quality values reported by the instrument; correcting the base quality scores reported by the instrument based on a model to obtain the corrected base quality scores.

2. The base quality scores correction method for sequencing platform characteristics according to claim 1, wherein in S1, the removing the low-quality sequences comprises: removing reads with a length less than 51 bp; sliding a window from the front to the back of the read with a window size of 1 base, and if an average base quality score in the window is ≤1, filtering out the bases on the right side of the window.

3. The base quality scores correction method for sequencing platform characteristics according to claim 2, wherein in S2, a consensus sequence of the reference genome is constructed, that is, bases with a mutation frequency ≥99% are taken as the true base types of a site.

4. The base quality scores correction method for sequencing platform characteristics according to claim 3, wherein in S2, the reads level filtering includes: filtering out read pairs with only one read aligned to the reference genome, read pairs not aligned to the reference genome, reads with non-primary alignments, reads that failed platform/supplier quality checks, and reads with embedded alignments but not primary representative alignments.

5. The base quality scores correction method for sequencing platform characteristics according to claim 4, wherein in S6, construction of the local weighted regression model is established by a lowess method of an sm.nonparametric module of Python, and a parameter frac is set to 0.1.

6. A base quality score correction device for sequencing platform characteristics, comprising:
an adapter removal module: used to remove adapter sequences from an original double-end sequencing data;
an alignment module: used to align an input sequencing data without adapters to a reference genome and generate a Bam file aligned to the reference genome;
a filtering module: used to construct a consensus sequence of the reference genome for the input Bam file generated and aligned to the reference genome and perform a reads-level filtering, wherein the consensus sequence of the reference genome is constructed, that is, a base with a mutation frequency greater than or equal to 99% is used as a true base type of a site; the reads-level filtering includes filtering out read pairs with only one read aligned to the reference genome, read pairs not aligned to the reference genome, reads with non-primary alignments, reads that failed the platform/supplier quality check, and reads with embedded alignments but not primary representative alignments, without filtering bases in overlapping part of read1 and read2 according to a base quality score, that is, the bases in the overlapping part of read1 and read2 are retained;
an overlapping base extraction and error source distinction module: used to extract all overlapping bases of read1 and read2 from the filtered Bam file, distinguish an error source according to a coordinate position on a same consensus sequence of the reference genome, whether the bases of read1 and read2 are complementary and paired, and whether the bases of read1 and read2 match the bases in the consensus sequence of the reference genome, the bases of read1 that are not paired with the bases of read2 and the bases that do not match the consensus sequence of the reference genome are marked as bases derived from sequencing errors;
an error rate statistics module: used to extract characteristics of the overlapping bases, including read direction, sequencing cycle number, dinucleotides composed of the previous base in a sequencing amplification direction, and the base quality score reported by an instrument, and divide the overlapping bases into different bins according to the characteristics, and count a base error rate in each bin; based on sequencing error bases in the bin, an empirical quality value is calculated according to Formula (I);
$$\{ \begin{matrix}empirical quality = - 10 * log 10 ( \frac{mismatches + 1}{bases + 2} ) & ( emperical quality <= 40 ) \\ empirical quality = 40 & ( emperical quality > 40 )\end{matrix} , \qquad Formula ( I )$$
in Formula (I), empirical quality represents the empirical quality value of the bin, mismatches represents a number of the sequencing error bases in the bin, and bases represents a number of all bases in the bin;
an correction model building module: used to fit and model the base quality score and the empirical quality values reported by the instrument under different characteristics using local weighted regression according to the empirical quality values calculated by the error rate statistics module; and
an correction module: used to correct the base quality score reported by the instrument according to a model established by the correction model building module, and output the corrected base quality scores.

7. An electronic device, comprising:
one or more processors; and
a storage device on which one or more programs are stored, and when the one or more programs are executed by the one or more processors, the one or more processors implement the base quality scores correction method for sequencing platform characteristics according to claim 1.
